(19) United States
(12) Patent Application Publication
(10) Pub. No.: US 2011/0104267 A1
(43) Pub. Date: May 5, 2011

(54) Pharmaceutical compositions of antiretrovirals

(75) Inventor: Umesh Mutt Hanjagi, AUROBINO PHARA (IN)

(73) Assignee: AUROBINDO PHARMA LIMITED, Hyderbad (IN)

(21) Appl. No.: 11/919,161

(22) PCT Filed: Apr. 24, 2006
(86) PCT No.: PCT/IB2006/001210

§ 371 Date: Sep. 15, 2009

(30) Foreign Application Priority Data
Apr. 25, 2005 (IN) 464/CHE/2005

(51) Int. Cl. A61K 31/7072; A61K 9/20; A61K 9/48; A61P 31/18

(52) U.S. Cl. 424/452; 514/50; 424/465

(57) ABSTRACT

The present invention relates to the stable pharmaceutical dosage forms of combination of antiretroviral agents. More particularly, the present invention relates to stable pharmaceutical dosage forms of Lamivudine, Zidovudine and Nevirapine, prepared by granulation technology.

### FIELD OF THE INVENTION

The present invention relates to the stable pharmaceutical dosage forms of combination of antiretroviral agents.

More particularly, the present invention relates to stable pharmaceutical dosage forms of Lamivudine, Zidovudine and Nevirapine, prepared by granulation technology.

### BACKGROUND OF THE INVENTION

The human immunodeficiency virus (HIV) is the causative agent of acquired immunodeficiency syndrome (AIDS). This disease is characterized by the destruction of the immune system, particularly of the CD4 and T-cell making the host susceptible to opportunistic infections. HIV is also associated with a precursor AIDS-related complex (ARC), a syndrome characterized by symptoms such as persistent generalized lymphadenopathy, fever and weight loss.

Anti-retroviral drugs, such as reverse transcriptase inhibitors and viral protease inhibitors, have been used to treat HIV infection. These treatments can effectively suppress viral production when used in combinations known as HAART (highly active anti-retroviral therapy).

Two general classes of reverse transcriptase inhibitors (RTIS) have been identified:

i) Nucleoside reverse transcriptase inhibitors (NRTIs). Eg: Zidovudine (AZT), Didanosine (DDI), Zalcitabine (ddC), Stavudine (d4T), Lamivudine (3TC) and Tenofovir (PMPA).
ii) Non-nucleoside reverse transcriptase inhibitors (NNRTIs). Eg: Efavirenz, Nevirapine and Delavirdine.

Chemically, Zidovudine is 3′-azido-3′-deoxythymidine, is a pyrimidine nucleoside analogue and is commercially available in various dosage forms such as tablets, capsules and oral solution under the trade name Retrovir®. Zidovudine, for treating HIV was first disclosed in U.S. Pat. No. 4,724,232.

Chemically, Lamivudine is (2R,cis)-4-amino-1-(2-hydroxymethyl-1,3-oxathiolan-5-yl)-(1H)-pyrimidin-2-one and is commercially available in tablets and oral solution under the trade names Epivir® and Epivir-HBV'. Lamivudine and method of treating HIV using Lamivudine was first disclosed in U.S. Pat. No. 5,047,407.

Chemically, Nevirapine is 11-cyclopropyl-5,11-dihydro-4-methyl-6H-dipyrido[3,2-b:2′,3′-e][1,4]diazepin-6-one and is commercially available in tablets and oral solution under the trade name Viramune®. Nevirapine and method of treating HIV with Nevirapine was first disclosed in U.S. Pat. No. 5,366,972.

The co-package of Lamivudine and Zidovudine plus Nevirapine has been tentatively approved by USFDA.

One substantial and persistent problem in the treatment of AIDS has been the ability of the HIV virus to develop resistance to the individual therapeutic agents employed to treat the disease. Thus, a need remains for an efficacious and long lasting therapy for AIDS which lowers HIV viral levels of patients to undetectable levels and raises CD4 cell counts for prolonged periods of time without the development of resistance.

Dosage forms containing single drugs of auto immuno deficiency syndrome [AIDS] are less long lasting therapy for AIDS and also has to take more number of dosage forms for treating AIDS. Hence, there is a need to develop combination of drugs to treat AIDS called as fixed dose combinations (FDC). FDC's are recommended in the world health organization [WHO] treatment guidelines and several generic FDC's have been pre-qualified by the WHO with a majority of them being marketed quite actively for some time now in a number of countries in Africa and Latin America.

Following are few patents/publications, discloses combinations of Antiretrovirals.
U.S. Pat. No. 5,627,186 discloses combination of Lamivudine and Zidovudine for treating HIV.
U.S. Pat. No. 6,417,191 discloses combination of Abacavir and Lamivudine; Abacavir, Lamivudine and Zidovudine for treating HIV.
WO 2004/089383 discloses combination of Lamivudine, Stavudine and Efavirenz for treating HIV.
WO 2004/089382 discloses combination of Lamivudine, Zidovudine and Efavirenz for treating HIV.
ZA 2001/10500 discloses pharmaceutical composition of Lamivudine, Zidovudine and Nevirapine and process, which comprises wet granulating Lamivudine, Zidovudine, Nevirapine and diluent with water; drying, sizing and blending the granules with disintegrant; lubricating the granules; and compressing the lubricated granules into tablets.
WO 2006/001029 discloses composition and process comprising granulating Zidovudine, Nevirapine, Lamivudine, microcrystalline cellulose, starch, croscarmellose sodium with a solution of polyvinylpyrrolidone k-30 and drying the granules, blending the dried granules with magnesium stearate, croscarmellose sodium, colloidal anhydrous silica, crospovidone and compressing the blend into tablets.

The above prior art discloses various fixed dose combinations (FDC) of antiretrovirals and their compositions. The various advantages of FDC's when compared to the separate ARV regimens are ease of use, better adherences to the dosage schedules, reduced risk of drug resistance and increased affordability and hence there is need to develop stable compositions for FDC's.

The above prior art discloses composition of Lamivudine, Zidovudine and Nevirapine and prepared by single granulation containing Lamivudine, Zidovudine and Nevirapine. The disclosed single granulation process may have bioequivalent problems when compare with individual drugs. Thus, the inventors of the present invention during their continuous efforts to develop bioequivalent composition of Lamivudine, Zidovudine and Nevirapine, developed a process, which involves granulating Lamivudine, Zidovudine and Nevirapine separately or granulating Lamivudine plus Zidovudine together and preparing granules of Nevirapine separately and further compressing the granules into tablets.

### OBJECTIVE OF THE INVENTION

Accordingly, the main objective of the present invention is to provide stable fixed dose combination of Lamivudine, Zidovudine and Nevirapine and process for preparing the fixed dose combination, using granulation technology.

Yet another objective of the present invention is to provide stable dosage forms of Lamivudine, Zidovudine and Nevirapine in such a way that it will comply with the reference product of each of these approved individual drugs in terms of in vitro parameters like dissolution, disintegration, etc and in vivo parameters like bioequivalence.

### SUMMARY OF THE INVENTION

According to the main embodiment, the present invention provides bioequivalent dosage form comprising Lamivudine, Zidovudine and Nevirapine prepared by granulation process comprising the steps of:

a) preparing granules comprising Lamivudine; Zidovudine and pharmaceutically acceptable excipients,
b) preparing granules of Nevirapine by granulating with pharmaceutically acceptable excipients,
c) blending the granules of step (a) and (b), with pharmaceutically acceptable excipients,
d) lubricating the blended granules and finally compressing the granules into tablets or tilled into capsules.

### DETAILED DESCRIPTION OF THE INVENTION

In an embodiment of the present invention, the granules of Lamivudine and Zidovudine may be prepared by a separate granulation involving granulation of lamivudine and zidovudine separately or granulating lamivudine and zidovudine in a single granulation process.

In another embodiment of the present invention, the granules of Lamivudine, Zidovudine and Nevirapine further comprise one or more pharmaceutical excipients.

In another embodiment of the present invention, the pharmaceutical excipients selected from diluents, binders, disintegrants, glidants and lubricants.

Suitable diluents used according to the present invention are selected from mannitol, lactose, microcrystalline cellulose, maltitol, maltodextrin, maltose, starch, calcium carbonate, calcium phosphate dibasic, calcium sulfate, and dextrates or a combination thereof.

Suitable binders used according to the present invention are selected from hydroxy propyl cellulose, hydroxypropyl methylcellulose, gelatin, hydroxy ethyl cellulose, povidone, starch and methylcellulose or a combination thereof.

Suitable disintegrants used according to the present invention are selected from sodium starch glycolate, croscarmellose sodium, crospovidone, hydroxypropyl cellulose, magnesium aluminum silicate, pregelatinized starch, cornstarch, sodium carboxymethyl cellulose, or a combination thereof.

Suitable glidants used according to the present invention are selected from magnesium trisilicate, talc, tribasic calcium phosphate, glyceryl monostearate, glyceryl stearate and silica dioxide or a combination thereof.

Suitable lubricants used according to the present invention are selected from calcium stearate, magnesium stearate, hydrogenated vegetable oil, stearic acid, magnesium aluminum silicate or a combination thereof.

The granules according to the present invention may be prepared by wet granulation or dry granulation such as compaction/slugging.

The wet granulation process according to the present invention comprises the steps of

a) granulating lamivudine and zidovudine or nveirapine and optionally filler, disintegrant, with solvent alone or a solution of binder,
b) drying the wet granules, and
c) sieving the dried granules to obtain uniform granules of lamivudine and zidovudine or nveirapine.

The solvents used according to the present invention may be selected from water, isopropyl alcohol, acetone or combination thereof.

The tablets according to present invention may optionally be coated to prevent the degradation of Lamivudine from light. The polymers used for coating selected from hydroxypropyl methylcellulose, hydroxypropyl cellulose, xanthan gum or a combination there of.

In another embodiment of the present invention, the solid dosage from may be in the form of tablets, bilayered tablets, and capsules.

The following examples further exemplify the invention and are not intended to limit the scope of the invention. It is obvious to those skilled in the art to find out the composition for other dosage forms and substitute the equivalent excipients as described in this specification or with the one known to the industry.

### Example 1

| S. No. | Ingredients | Quantity per unit (mg) |
|---|---|---|
| 1 | Lamivudine | 150.00 |
| 2 | Zidovudine | 300.00 |
| 3 | Nevirapine Anhydrous | 200.00 |
| 4 | Microcrystalline Cellulose | 192.00 |
| 5 | Sodium Starch Glycolate | 4.00 |
| 6 | Lactose Monohydrate | 120.00 |
| 7 | Povidone | 3.00 |

Extragranular ingredients

| | | |
|---|---|---|
| 8 | Sodium Starch Glycolate | 15.00 |
| 9 | Colloidal Silicone Dioxide | 8.00 |
| 10 | Magnesium Stearate | 8.00 |

| | |
|---|---|
| Tablet Weight | 1000 |

Film Coating

| | | |
|---|---|---|
| 11 | Opadry 13B58802 | 30.00 |

The processing steps involved were:
a) preparation of granules of Lamivudine plus Zidovudine:
i) sifted and blended Lamivudine, Zidovudine, microcrystalline cellulose,
ii) granulated the blend using water,
iii) dried and sized the wet mass,
b) preparation of granules of Nevirapine:
i) sifted and blended Nevirapine, sodium starch glycolate, microcrystalline cellulose and lactose monohydrate,
ii) granulated the blend using povidone,
iii) dried and sized wet mass,
c) blending and lubrication of Lamivudine-Zidovudine granules and Nevirapine granules:
i) sifted and blended sodium starch glycolate, magnesium stearate, colloidal silicone dioxide,
ii) blended the granules of Lamivudine plus Zidovudine obtained in step (a) and granules of Nevirapine obtained in step (b) with the blend of step (i),
iii) lubricated the blend of step (ii) with magnesium stearate and compressed into tablets or filled into capsules and
iv) tablets were coated with film coating polymers.

### Example 2

| S. No | Ingredients | Quantity per unit (mg) |
|---|---|---|
| 1 | Lamivudine | 150.0 |
| 2 | Zidovudine | 300.0 |
| 3 | Microcrystalline Cellulose | 250.0 |
| 4 | Nevirapine | 200.0 |
| 5 | Lactose monohydrate | 47.0 |
| 6 | Povidone K30 | 3.0 |
| 8 | Purified water | qs |

Extragranular stage

| | | |
|---|---|---|
| 10 | Sodium starch Glycolate | 30.0 |
| 11 | Colloidal Silicon Dioxide | 12.5 |
| 12 | Magnesium Stearate | 7.5 |
| | Tablet weight (mg) | 1000.0 |

Coating

| | | |
|---|---|---|
| 13 | Opadry 13B58802 White IH | 30.00 |
| 14 | Purified water USP | q.s. |
| | Coated Tablet weight | 1030.00 |

The processing steps involved were:
a) preparation of granules of Lamivudine plus Zidovudine:
i) sifted and blended Lamivudine, Zidovudine, microcrystalline cellulose,
ii) granulated the blend using water,
iii) dried and sized the wet mass,
b) preparation of granules of Nevirapine:
i) sifted and blended Nevirapine, microcrystalline cellulose and lactose monohydrate,
ii) granulated the blend using povidone,
iii) dried and sized wet mass,
c) blending and lubrication of Lamivudine-Zidovudine granules and Nevirapine granules:
i) sifted and blended sodium starch glycolate, magnesium stearate, colloidal silicone dioxide,
ii) blended the granules of Lamivudine plus Zidovudine obtained in step (a) and granules of Nevirapine obtained in step (b) with the blend of step (i),
iii) lubricated the blend of step (ii) with magnesium stearate and compressed into tablets or filled into capsules and
iv) tablets were coated with film coating polymers.

### Example 3

| | | |
|---|---|---|
| S. No. | Ingredients | Quantity per unit (mg) |

Granulation I
Intragranular stage

| | | |
|---|---|---|
| 1 | Lamivudine | 150.0 |
| 2 | Zidovudine | 300.0 |
| 3 | Microcrystalline Cellulose | 114.5 |
| 4 | Sodium starch Glycolate | 10.0 |
| 5 | Purified water | qs |

Extragranular stage

| | | |
|---|---|---|
| 6 | Sodium starch Glycolate | 11.0 |
| 7 | Colloidal Silicon Dioxide | 5.0 |
| 8 | Magnesium Stearate | 5.0 |

Granulation II
Intragranular stage

| | | |
|---|---|---|
| 9 | Nevirapine | 200.0 |
| 10 | Lactose monohydrate | 100.0 |
| 11 | Microcrystalline Cellulose | 86.5 |
| 12 | Povidone K30 | 3.0 |
| 13 | Quinoline Yellow | 0.5 |
| 14 | Purified water | qs |

Extragranular stage

| | | |
|---|---|---|
| 15 | Sodium starch Glycolate | 8.0 |
| 16 | Colloidal Silicon Dioxide | 3.5 |
| 17 | Magnesium Stearate | 3.0 |

The processing steps involved were:
a) preparation of granules of Lamivudine plus Zidovudine:
i) sifted and blended Lamivudine, Zidovudine, sodium starch glycolate microcrystalline cellulose,
ii) granulated the blend using water,
iii) dried and sized the wet mass,
iv) dried granules were blended with sodium starch glycolate, colloidal silicon dioxide,
v) lubricated the blended granules with magnesium stearate.
b) preparation of granules of Nevirapine:
i) sifted and blended Nevirapine, microcrystalline cellulose and lactose monohydrate,
ii) granulated the blend using povidone,
iii) dried and sized wet mass,
iv) dried granules were blended with sodium starch glycolate, colloidal silicon dioxide,
v) lubricated the blended granules with magnesium stearate.
c) preparation of bilayered tablets or capsules:
i) blend (a) and (b) are filled into capsules or compressed into bilayered tablets.
iv) tablets were optionally coated with film coating polymers.

### Example 4

| S. No. | Ingredients | Quantity per unit (mg) |
|---|---|---|
| 1 | Lamivudine | 150.00 |
| 2 | Zidovudine | 300.00 |
| 3 | Nevirapine Anhydrous | 200.00 |
| 4 | Microcrystalline Cellulose | 184.00 |
| 5 | Sodium Strach Glycollate | 4.00 |
| 6 | Lactose Monohydrate | 120.00 |
| 7 | Povidone | 3.00 |
| 8 | Hypromellose | 8.00 |

Extragranular ingredients

| | | |
|---|---|---|
| 9 | Sodium Starch Glycolate | 15.00 |
| 10 | Colloidal Silicone Dioxide | 8.00 |
| 11 | Magnesium Stearate | 8.00 |

| | |
|---|---|
| Tablet weight | 1000 |

Film Coating

| | | |
|---|---|---|
| 12 | Opadry 13B58802 | 30.00 |

The processing steps involved were:
[a] microcrystalline cellulose was sifted through a suitable mesh and divided into 3 equal parts,
b) preparation of granules of Lamivudine:
i) sifted and blended Lamivudine, microcrystalline cellulose,
ii) wet granulated the blend using water,
iii) dried and sized the wet mass,
c) preparation of granules of Zidovudine:
i) sifted and blended Zidovudine and microcrystalline cellulose,
ii) granulated the blend using hypromellose,
iii) dried and sized the wet mass,
d) preparation of granules of Nevirapine:
i) sifted and blended Nevirapine, sodium starch glycolate, lactose monohydrate and microcrystalline cellulose,
ii) granulated the blend using povidone.
iii) dried and sized the wet mass.
e) blending and lubrication of Lamivudine, Zidovudine and Nevirapine granules:
i) sifted and blended sodium starch glycolate, magnesium stearate, colloidal silicon dioxide,
ii) blended the granules of Lamivudine obtained in Step (b), Zidovudine obtained in step (c) and Nevirapine obtained in step (d) with blend of step (i),
iii) lubricated the blend of step (ii) with magnesium stearate and compressed into tablets or filled into capsules and
iv) tablets were coated with film coating polymers.

### Dissolution Profile of Lamivudine, Zidovudine and Nevirapine Tablets

The tablets were subjected to an in vitro dissolution method to determine the rate at which the Lamivudine, Zidovudine and Nevirapine was released from the tablets. The tablets were placed into a dissolution medium of 0.01 N HCl and stirred with paddles at 50 rpm (USP 2 apparatus). The dissolution profile is given in Table 1.

TABLE 1

| | (%) Release | | |
|---|---|---|---|
| Time | Lamivudine | Zidovudine | Nevirapine |

Example 2

| | | | |
|---|---|---|---|
| 5 min | 89 | 88 | 57 |
| 10 min | 92 | 92 | 68 |
| 15 min | 94 | 94 | 75 |
| 30 min | 96 | 96 | 83 |
| 45 min | 97 | 97 | 86 |
| 60 min | 98 | 98 | 88 |

Example 3

| | | | |
|---|---|---|---|
| 5 min | 90 | 89 | 56 |
| 10 min | 93 | 93 | 69 |
| 15 min | 94 | 94 | 75 |
| 30 min | 95 | 95 | 83 |
| 45 min | 97 | 97 | 88 |
| 60 min | 97 | 97 | 91 |

Example 4

| | | | |
|---|---|---|---|
| 5 min | 78 | 54 | 52 |
| 10 min | 91 | 73 | 69 |
| 15 min | 93 | 81 | 76 |
| 30 min | 96 | 89 | 83 |
| 45 min | 96 | 91 | 86 |
| 60 min | 96 | 93 | 87 |

1. A stable solid dosage form comprising Lamivudine, Zidovudine and Nevirapine prepared by granulation process comprising the steps of:
a) preparing granules comprising Lamivudine, Zidovudine and pharmaceutically acceptable excipients,
b) preparing granules of Nevirapine by granulating with pharmaceutically acceptable excipients,
c) blending the granules of step (a) and (b), with pharmaceutically acceptable excipients,
d) lubricating the blended granules and finally compressing the granules into tablets or filled into capsules.

2. The granules of Lamivudine and Zidovudine as claimed in claim 1, prepared by a separate granulation involving granulation of lamivudine and zidovudine separately.

3. The granules of Lamivudine and Zidovudine as claimed in claim 1, prepared by granulating lamivudine and zidovudine in a single granulation process.

4. The granules as claimed in claim 1, further comprise one or more pharmaceutical excipients such as diluents, binders, disintegrants, glidants and lubricants.

5. The granules as claimed in claim 4, wherein the diluent is selected from mannitol, lactose, microcrystalline cellulose, maltitol, maltodextrin, maltose, starch, calcium carbonate, calcium phosphate dibasic, calcium sulfate, and dextrates or a combination thereof.

6. The granules as claimed in claim 4, wherein the binder is selected from hydroxy propyl cellulose, hydroxypropyl methylcellulose, gelatin, hydroxy ethyl cellulose, povidone, starch and methylcellulose or a combination thereof.

7. The granules as claimed in claim 4, wherein the disintegrant is selected from sodium starch glycolate, croscarmellose sodium, crospovidone, hydroxypropyl cellulose, magnesium aluminum silicate, pregelatinized starch, cornstarch, sodium carboxymethyl cellulose or a combination thereof.

8. The granules as claimed in claim 4, wherein the glidant is selected from magnesium trisilicate, talc, tribasic calcium phosphate, glyceryl monostearate, glyceryl stearate and silica dioxide.

9. The granules as claimed in claim 4, wherein the lubricant is selected from calcium stearate, magnesium stearate, hydrogenated vegetable oil, stearic acid, magnesium aluminum silicate or a combination thereof.

10. The granules as claimed in claim 1, prepared by wet granulation or dry granulation.

11. The wet granulation process as claimed in claim 10, comprising the steps of
a) granulating lamivudine and zidovudine and optionally filler, disintegrant, with solvent alone or a solution of binder,
b) drying the wet granules and
c) sieving the dried granules to obtain uniform granules of lamivudine and zidovudine.

12. The wet granulation process as claimed in claim 10, comprising the steps of
a) granulating Nevirapine and filler, disintegrant, with solvent alone or a solution of binder,
b) drying the wet granules and
c) sieving the dried granules to obtain uniform granules of Nevirapine.

13. The solvent as claimed in claim 11, is selected from water, isopropyl alcohol, acetone or combination thereof.

14. The stable dosage form as claimed in claim 1, is in the form of a tablet, bi layered tablet or capsule.
